(19) United States
(12) Patent Application Publication
(10) Pub. No.: US 2020/0238254 A1
(43) Pub. Date: Jul. 30, 2020

(54) FUNCTIONALIZED HIGH-DENSITY CHROMATOGRAPHY MATRIX, PREPARATION METHOD AND APPLICATION THEREOF

(71) Applicant: Dalian University of Technology, Dalian, Liaoning (CN)

(72) Inventors: Fang CHENG, Dalian, Liaoning (CN); Mingyang LI, Dalian, Liaoning (CN); Wei HE, Dalian, Liaoning (CN); Bingbing SUN, Dalian, Liaoning (CN); Xiaochun MA, Dalian, Liaoning (CN); Jingping QU, Dalian, Liaoning (CN)

(21) Appl. No.: 16/486,765

(22) PCT Filed: May 18, 2018
(86) PCT No.: PCT/CN2018/087455

§ 371 Date: Aug. 16, 2019

(30) Foreign Application Priority Data
Apr. 2, 2018 (CN) 201810283350.1

(51) Int. Cl. B01J 20/26; B01J 20/28; C07K 1/22

(52) U.S. Cl. CPC B01J 20/265; B01J 20/262; B01J 20/327; B01J 20/28047; C07K 1/22; B01J 20/28019

(57) ABSTRACT

In a method to functionalize high-density chromatography matrix functionalization method, the hydrophilic microsphere having a surface polyhydroxy structure or the hydrophilic microsphere coated with a polyhydroxy polymer is used as chromatography matrix, under anhydrous conditions with catalyst, catalyze surface hydroxyl group of the matrix to react with divinyl sulfone, and achieve functionalization of the hydroxyl group on the surface of the matrix. The chromatography matrix surface functionalization method of the present disclosure has few steps, simple process and high functional density, and the reagents and solvents used are conventional reagents and can be recycled and reused. No byproduct is generated during the reaction with high atom economy and low cost, and the density control can be achieved by adjusting the reaction time and catalyst type.

### TECHNICAL FIELD

The present disclosure belongs to the technical field of chromatographic material preparation of biochemical industry, and in particular relates to a functionalized high-density chromatography matrix, its preparation method and application thereof.

### BACKGROUND ART

Antibodies and related products have important roles in the clinical treatment and immunological diagnosis. In recent years, with the rapid development of cell culture technology, there are increasing requirements for the purification process of antibody. The chromatography matrix activation method with high reactivity and controllable reaction is of great significance for increasing the adsorption capacity of the matrix and optimizing the adsorption specificity. At present, the widely used activating agents for chromatography matrixes are epichlorohydrin/epibromohydrin and allyl bromide. Harding et al. (J. Chromatogr. A, 1997, 775: 29) studied the functionalization effects of epichlorohydrin/epibromohydrin and allyl bromide on different polyhydroxyl chromatography matrices in different conditions. Currently, Pall Biosepara has commercially produced chromatographic matrices based on the above functionalization method with 4-pyridylethylmercaptan as a ligand. However, this method is complicated with high cost, and its reaction conditions are harsh and a strong alkali condition is required; in addition, hydrogen halide by-products may be produced during the reactions, with poor atom economy.

In 1975, Porath et al. disclosed the use of divinyl sulfone (DVS) as a functionalized reagent of chromatography matrix (Porath et al. J. Chromatogr. A, 1975, 73: 1767). This functionalization method has the following advantages: there is no generation of by-products; the reacted vinyl sulfonyl (VS) group can be efficiently linked to a ligand containing sulfydryl, amidogen and hydroxyl, with wide applicability; sulfone groups can provide a thiophilic action during antibody binding and enhance the selectivity of antibody binding. Application CN 101284224A disclosed an expanded bed adsorption matrix, prepared by this functionalization method, for isolating antibodies. However, the chromatography matrix functionalization using divinyl sulfone is carried out in alkaline aqueous solution with low reaction efficiency, its highest functional density is only 60 μmol/g resin, and the reaction is uncontrollable, which restricts the improvement of adsorption capacity and adsorption specificity of chromatography matrix. In addition, the vinylsulfonly is easily hydrolyzed under the reaction conditions of alkaline aqueous solution, reducing the amount of ligand coupling. Therefore, it is of great significance to improve the efficiency of functionalization of divinyl sulfone to achieve high-density vinyl sulfone group functionalization with controllable chromatography matrix density.

### SUMMARY OF THE INVENTION

The object of the present disclosure is to provide a functionalization method for a high-density polyhydroxyl chromatography matrix with high reaction efficiency and controllable density. The present disclosure adopts the following technical solutions:

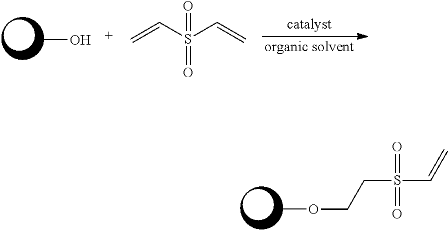

A method for preparing functionalized high-density chromatography matrix with controllable density, comprising the following steps:

dissolving catalyst and divinyl sulfone in organic solvent, adding chromatography matrix removed water to obtain a reaction solution, and reacting 0-48 h at 15-60° C., obtaining a functionalized chromatography matrix; wherein the catalyst is pyridine derivatives or trisubstituted organic phosphorus compounds.

In the present technical solution, the catalyst is selected from triphenylphosphine, tricyclohexylphosphine, triisopropylphosphine, trimethylphenylphosphine, tri-p-tolylphosphine, triphenylphosphine tri-sulfonate, pyridine, pyridinedicarboxylic acid and 4-dimethylaminopyridine.

In the present technical solution, the organic solvent is an aprotic solvent. Preferably, the organic solvent is selected from dichloromethane, acetone, acetonitrile, dimethyl sulfoxide or dimethylformamide.

In the present technical solution, the chromatography matrix is hydrophilic microsphere having surface polyhydroxy structure or hydrophilic microsphere which surface being coated with polyhydroxy polymer. Preferably, the chromatography matrix is agarose gel or microsphere coated with PVA, dextran or cellulose.

In the present technical solution, the molar ratio of the catalyst to divinyl sulfone is from 1:10 to 1:1000, preferably from 1:10 to 1:100.

In the present technical solution, the reaction temperature is from 15 to 45° C., preferably from 20 to 35° C.

In the present technical solution, preferably, the method further comprises a suction filtration and washing process after reaction at 15-60° C. for 0-48 h, and finally a functionalized chromatography matrix is obtained.

In the present technical solution, the reaction at 15-60° C. for 0-48 h means that the reaction is carried out in a constant temperature shaking table at 300-1000 rpm, and a good effect can be achieved within the reaction time of 0-24 h.

Usually the chromatography matrix can be completely dehydrated by using organic solvent; a person skilled in the art can also select a suitable dehydration method according to the following request: 1, it can thoroughly remove water in the chromatography matrix; and 2, it will not cause damage to the chromatographic matrix.

In the present technical solution, the concentration of the divinyl sulfone in the organic solvent is 1-20% (v/v).

In the present technical solution, the final concentration of the chromatography matrix is 0.1-0.3 g/mL in the reaction solution.

In addition, the present invention further protects a chromatography matrix prepared by the method described above in the present disclosure, and the applications of the chromatography matrix in the preparation of packing of affinity chromatography and purification of antibody drugs. In detailed, the preparation of packing of affinity chromatography means that the matrix activated by the method of the present disclosure can be coupled with a plurality of affinity ligands, and theoretically, coupling can be achieved as long as the ligands contain sulfydryl, amino and hydroxyl group, for example, the commonly used nitrilotriacetic acid (NTA), polypeptides (glutathione, etc.), polysaccharides, etc.

Beneficial Effects:

(1) High density: for the chromatography matrix prepared by the method described above, preferably triphenylphosphine is used as a catalyst, and the molar ratio of triphenylphosphine to divinyl sulfone is 1:10, and the concentration of vinyl sulfone in the organic solvent is 10% (v/v), and the concentration of the chromatography matrix is 0.1 g/mL; and the mixtures are reacted for 12 hours at 25° C. in a constant temperature shaking table at 1000 rpm. The surface has a density of vinyl sulfone group of up to 200 μmol/g, which is three times higher than that of conventional methods.

(2) Controllable density: the chromatography matrix prepared by the method described above can adjust its surface vinyl sulfone group density by adjusting the catalytic reaction time and the kinds of catalysts, and the density is adjusted within the range of 60-200 μmol/g, that is, different kinds of catalysts have different catalytic efficiency, and under the same reaction conditions, the surface vinylsulfonly has different density by using different catalysts. The catalytic reaction conforms to the first-order reaction kinetics, and the density of surface vinylsulfonly is different when catalyzed with the same catalyst for different time.

(3) High reaction efficiency: the chromatography matrix prepared by the method in the present disclosure has high reaction efficiency, and the reaction rate constant thereof can reach 0.011 min⁻¹.

(4) Simple operation, low cost and high atom economy: the method for preparing functionalized high-density chromatography matrix with controllable density of the present disclosure includes few steps and the operation method thereof is simple. The reagents and solvents used are conventional reagents and can be recycled. There are no by-products generated during the reaction, with high atom economy and low cost.

### BRIEF DESCRIPTION OF THE DRAWINGS

The present disclosure has 5 drawings which are intended to be illustrative of the present disclosure and constitute a part of the description. The drawings and following embodiments are used to construe the present disclosure instead of limiting it.

FIG. 1 shows the results of X-ray Photoelectron Spectroscopy (XPS) of the resin modified in each step in Embodiment 5. Wherein, FIG. 1 panel (a) represents a full spectrum; FIG. 1 panel (b) represents a S 2p spectrum; FIG. 1 panel (c) represents a C 1s spectrum. And curve (1) is an unmodified agarose gel; curve (2) is an agarose gel after VS functionalization reaction using a catalyst.

FIG. 2 shows the surface VS density change of the resin which repeated functionalization modification by using the three methods in Embodiment 6.

FIG. 3 shows a static adsorption curve of the resin in Embodiment 7 to Human IgG.

FIG. 4 shows a chromatogram of monoclonal antibody stock solution purified by resin in Embodiment 9. Wherein, curve (1) is agarose resin modified in Embodiment 7; curve (2) is MEP HyperCel; curve (3) is agarose gel modified under alkaline conditions. Part (a) represents the loading step; part (b) represents the elution step of acetate buffer at pH 4; part (c) represents the on-line washing step of 0.1 M NaOH solution.

FIG. 5 shows a GPC spectrum of monoclonal antibody stock solution purified by resin in Embodiment 9. Wherein, curve 1 represents a monoclonal antibody purified by the agarose resin, the agarose resin is the one modified in Embodiment 7; curve 2 represents a monoclonal antibody purified by MEP HyperCel; and 3 represents a monoclonal antibody purified by the agarose gel, the agarose gel is the one modified under alkaline conditions. Curve B represents acetate buffer at pH 4; curve S represents a monoclonal antibody purified by commercially-available ProteinA resin; curve F represents an unpurified monoclonal antibody stock solution.

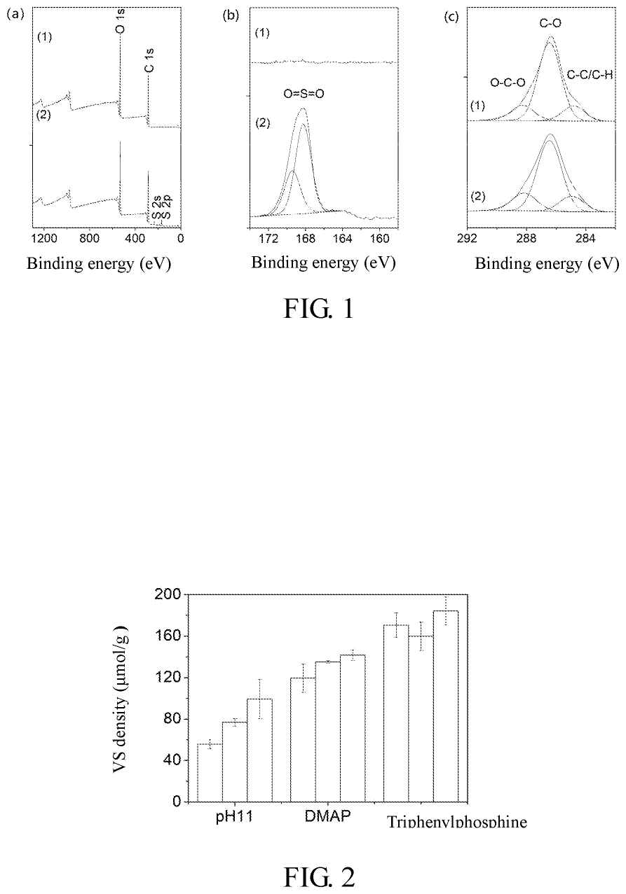

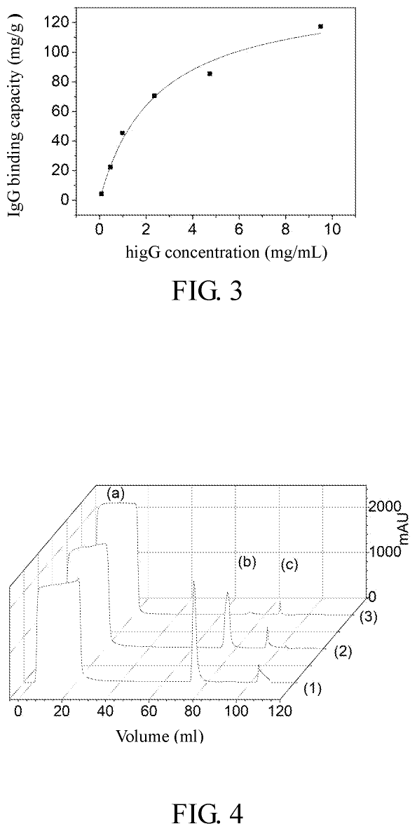

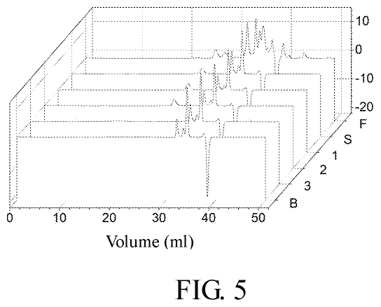

### DETAILED DESCRIPTIONS OF THE EMBODIMENTS

The following non-limiting embodiments are provided to enable those skilled in the art to fully understand the present disclosure, but not to limit the present disclosure in any way.

### Embodiment 1

0.3 g of agarose resin was taken (Bastarose 6FF: highly crosslinked 6% agarose, average particle size 90 μm; Bestchrom Biosciences Co., Ltd.), and suction filtered and thoroughly washed with acetonitrile to remove water, then 1 mL of 10% (v/v) divinyl sulfone in acetonitrile solution containing 4-dimethylaminopyridine was added, wherein the molar ratio of 4-dimethylaminopyridine to divinyl sulfone was 1:10, and reacted for 12 h at 25° C. After reaction, the solution was suction filtered and washed with acetonitrile to thoroughly remove divinyl sulfone and catalyst residue, to obtain a VS functionalized agarose resin.

In the control group, 0.3 g of agarose resin was added to 1 mL of carbonate buffer (0.5 M, pH 11), the carbonate buffer contains 10% (v/v) divinyl sulfone and 10% acetone, and reacted at 25° C. for 12 h. After reaction, the solution was suction filtered and washed with acetonitrile to thoroughly remove the divinyl sulfone residue. A certain amount of the functionalized agarose resin was taken, and excessive cysteine solution was added to fully react with the surface VS, and the change of the cysteine in the solution before and after the reaction was measured by the Ellman method, thereby the VS density of the resin surface was obtained. By calculating, the functional density obtained by the catalytic method of the present disclosure could reach 120 μmol/g. The density was twice of that obtained by conventional alkaline conditions.

### Embodiment 2

0.3 g of agarose resin was taken (Bastarose 6FF: highly crosslinked 6% agarose, average particle size 90 μm; Bestchrom Biosciences Co., Ltd.), and suction filtered and thoroughly washed with acetonitrile to remove water, then 1 mL of 10% (v/v) divinyl sulfone in acetonitrile solution containing triphenylphosphine was added, wherein the molar ratio of triphenylphosphine to divinyl sulfone was 1:10, and reacted for 1 h at 25° C. After reaction, the solution was suction filtered and washed with acetonitrile to thoroughly remove divinyl sulfone and catalyst residue, to obtain a VS functionalized agarose resin.

In the control group, 0.3 g of agarose resin was added to 1 mL of carbonate buffer (0.5 M, pH 11), the carbonate buffer contains 10% (v/v) divinyl sulfone and 10% acetone, and reacted at 25° C. for 12 h. After reaction, the solution was suction filtered and washed with acetonitrile to thoroughly remove the divinyl sulfone residue. A certain amount of the functionalized agarose resin was taken, and excessive cysteine solution was added to fully react with the surface VS, and the change of the cysteine in the solution before and after the reaction was measured by the Ellman method, thereby the VS density of the resin surface was obtained. By calculating, the functional density obtained could reach 80 μmol/g. The density was 1.3 times of that obtained by conventional alkaline conditions.

### Embodiment 3

0.3 g of agarose resin was taken (Bastarose 6FF: highly crosslinked 6% agarose, average particle size 90 μm; Bestchrom Biosciences Co., Ltd.) and suction filtered and thoroughly washed with acetonitrile to remove water, then 1 mL of 10% (v/v) divinyl sulfone in acetonitrile solution containing triphenylphosphine was added, wherein the molar ratio of triphenylphosphine to divinyl sulfone was 1:10, and reacted for 2 h at 25° C. After reaction, the solution was suction filtered and washed with acetonitrile to thoroughly remove divinyl sulfone and catalyst residue, to obtain a VS functionalized agarose resin.

In the control group, 0.3 g of agarose resin was added to 1 mL of carbonate buffer (0.5 M, pH 11), the carbonate buffer contains 10% (v/v) divinyl sulfone and 10% acetone, and reacted at 25° C. for 12 h. After reaction, the solution was suction filtered and washed with acetonitrile to thoroughly remove the divinyl sulfone residue. A certain amount of the functionalized agarose resin was taken, and excessive cysteine solution was added to fully react with the surface VS, and the change of the cysteine in the solution before and after the reaction was measured by the Ellman method, thereby the VS density of the resin surface was obtained. By calculating, the functional density obtained could reach 120 μmol/g. The density was twice of that obtained by conventional alkaline conditions.

### Embodiment 4

0.3 g of agarose resin was taken (Bastarose 6FF: highly crosslinked 6% agarose, average particle size 90 μm; Bestchrom Biosciences Co., Ltd.) and suction filtered and thoroughly washed with acetonitrile to remove water, then 1 mL of 10% (v/v) divinyl sulfone in acetonitrile solution containing triphenylphosphine was added, wherein the molar ratio of triphenylphosphine to divinyl sulfone was 1:10, and reacted for 12 h at 25° C. After reaction, the solution was suction filtered and washed with acetonitrile to thoroughly remove divinyl sulfone and catalyst residue, to obtain a VS functionalized agarose resin.

In the control group, 0.3 g of agarose resin was added to 1 mL of carbonate buffer (0.5 M, pH 11), the carbonate buffer contains 10% (v/v) divinyl sulfone, and reacted at 25° C. for 12 h. After reaction, the solution was suction filtered and washed with acetonitrile to thoroughly remove the divinyl sulfone residue. A certain amount of the functionalized agarose resin was taken, and excessive cysteine solution was added to fully react with the surface VS, and then the change of the cysteine in the solution before and after the reaction was measured by the Ellman method, thereby the VS density of the resin surface was obtained. By calculating, the functional density obtained could reach 160 μmol/g. The density was 2.7 times of that obtained by conventional alkaline conditions.

### Embodiment 5

The agarose resin was functionalized according to the procedure described in Embodiment 4. The resins before and after the functionalization were freeze-dried and characterized by XPS (X-ray photoelectron spectroscopy). No S (sulfur) element was detected on the surface of the agarose resin which was not functionalized. After VS functionalization, the S 2p peak was detected at 169 eV, which belonged to the sulfone group peak. It means that the VS functionalization was successfully achieved under the catalyst conditions.

### Embodiment 6

Triphenylphosphine and 4-dimethylaminopyridine were selected as catalysts respectively, and the functionalization condition of pH 11 was used as a control. Three times of repetitive functional modifications of agarose resin were performed according to the methods described in Embodiment 1 and Embodiment 4 respectively, and the VS density after each reaction was detected. Results show that the resin functionalized by the catalyst method of the present disclosure reached the highest density at the first functional modification, and the VS density did not increase significantly in the subsequent repeated modification steps. However, the VS density of resins with pH 11 functionalization condition increased significantly after each repetitive modification. It means that the catalyst method of the present disclosure has higher reaction efficiency than conventional methods.

### Embodiment 7

0.2 g of the functionalized agarose resin obtained by the method described in Embodiment 4 was added to 1 mL of HEPEs buffer (20 mM, pH 8.0) containing 10 mg/mL MEP, and reacted at 25° C. for 6 h to obtain MEP-modified agarose resin. The static adsorption performance of IgG was tested by using the resins.

The resin was first washed with deionized water and equilibrated with buffer. 0.04 g resin was accurately weighed and placed into a 2 mL centrifuge tube, and then 1 mL buffer solution of human IgG at different concentrations was added respectively and adsorbed for 3 h at a constant temperature of 25° C. After reaching the adsorption equilibrium, the solution was centrifuged and separated, and the supernate was taken out to determine the concentration of human IgG. The adsorption capacity of the resin was calculated according to material balance, and the adsorption isotherm was drawn, and then the saturated adsorption capacity and dissociation constant were obtained by fitting according to the Langmuir equation. The resin, which prepared in Embodiment 4, modified by MEP of the present disclosure had a saturated adsorption capacity of 141.4 mg/g • resin for human IgG, and a dissociation constant of 1.61×10⁻⁵M⁻¹.

### Embodiment 8

The dynamic binding capacity of human IgG (Wako Pure Chemical Industries, Ltd.) of the MEP-modified resin obtained in Embodiment 7 was tested. 1 mL of the resin was loaded into a 1 mL column to calculate the dynamic binding capacity at 10% penetration at different flow rates respectively. The dynamic binding capacity of the resin prepared in Embodiment 1 was 84.4 mg/g • resin at 10% penetration of human IgG at a flow rate of 0.25 mL/min, and the dynamic binding capacity of the resin was 29.6 mg/g • resin at 10% penetration of human IgG at a flow rate of 0.5 mL/min, and the dynamic binding capacity of the resin was 15.9 mg/g • resin at 10% penetration of human IgG at a flow rate of 1.0 mL/min.

### Embodiment 9

The monoclonal antibody (omalizumab) in the serum-free cell culture supernatant was purified by the MEP-modified resin obtained in Embodiment 7, and the resin obtained by reacting the commercial product MEP HyperCel (PALL) under conventional alkaline conditions was used as a control. 1 mL of resin was loaded into a 1 mL column at a flow rate of 0.5 mL/min, and eluted with 20 mM sodium acetate buffer at a pH of 4, and finally washed in situ with 0.1 M NaOH. The eluted samples were analyzed by GPC. The purity of the monoclonal antibody purified by the resin prepared in Embodiment 7 was higher than 95%, and the purified amount was 1.43 times of that of MEP HyperCel.

Many possible variations and modifications may be made to the technical solutions of the present disclosure by using the technical contents disclosed above or equivalent embodiments are made by those skilled in the art without departing from the scope of technical solutions of the present invention. Therefore, any simple modifications, equivalent changes and modifications of the above embodiments made according to the technical essence of the present invention without departing from the technical solutions of the present invention shall fall into the scope of protection of the present invention.

1. A method for preparing functionalized high-density chromatography matrix, comprising the following steps: dissolving catalyst and divinyl sulfone in organic solvent, adding chromatography matrix removed water to obtain a reaction solution, and reacting 0-48 h at 15-60° C., obtaining a functionalized chromatography matrix; wherein the catalyst is pyridine derivatives or trisubstituted organic phosphorus compounds.

2. The method according to claim 1, wherein the catalyst is selected from triphenylphosphine, tricyclohexylphosphine, triisopropylphosphine, trimethylphenylphosphine, tri-p-tolylphosphine, triphenylphosphine tri-m-sulfonate, pyridine, pyridinedicarboxylic acid and 4-dimethylaminopyridine.

3. The method according to claim 1, wherein the organic solvent is aprotic solvent.

4. The method according to claim 3, wherein the organic solvent is dichloromethane, acetone, acetonitrile, dimethyl sulfoxide or dimethylformamide.

5. The method according to claim 1, wherein the chromatography matrix is hydrophilic microsphere having surface polyhydroxy structure.

6. The method according to claim 5, wherein the chromatography matrix is agarose gel or microsphere coated with PVA, dextran or cellulose.

7. The method according to claim 1, wherein the molar ratio of the catalyst to divinyl sulfone is from 1:10 to 1:1000.

8. The method according to claim 1, wherein the reaction temperature is from 15 to 45° C.

9. The method according to claim 1, wherein the concentration of the divinyl sulfone in the organic solvent is 1-20% (v/v).

10. The method according to claim 1, wherein the final concentration of the chromatography matrix is 0.1-0.3 g/mL in the reaction solution.

11. A chromatography matrix prepared by the method described according to claim 1.

12. Applications of the chromatography matrix prepared according to claim 1 in the preparation of packing of affinity chromatography and purification of antibody drugs.
